(19) United States
(12) Patent Application Publication
(10) Pub. No.: US 2024/0215889 A1
(43) Pub. Date: Jul. 4, 2024

(54) THIN FILM ENDOVASCULAR ELECTRODE ARRAY AND METHOD OF FABRICATION

(71) Applicant: University of Southern California, Los Angeles, CA (US)

(72) Inventors: Brianna Leilani THIELEN, Los Angeles, CA (US); Ellis MENG, La Canada Flintridge, CA (US)

(73) Assignee: University of Southern California, Los Angeles, CA (US)

(21) Appl. No.: 18/401,817

(22) Filed: Jan. 2, 2024

Related U.S. Application Data
(60) Provisional application No. 63/436,301, filed on Dec. 30, 2022.

(51) Int. Cl. A61B 5/263; A61B 5/00

(52) U.S. Cl. CPC A61B 5/263; A61B 5/6851; A61N 1/37516

(57) ABSTRACT

An endovascular device includes a thin film polymer strip having an electrode array. The thin film polymer strip includes a thin film polymer shaped into a strip having a distal end and a proximal end and a plurality of exposed metal electrodes positioned at the distal end of the thin film polymer. Each metal electrode is composed of a metal film. The thin film polymer strip also includes a plurality of bond pads positioned at the proximal end of the thin film polymer. A plurality of insulated traces connect each electrode to a single bond pad. The thin film polymer strip also includes insulated micro-wires connected to bond pads and recording or stimulation equipment. Characteristically, the thin film polymer strip has a helical or cylindrical shape configured to fit around a commercial vascular guide wire.

### CROSS-REFERENCE TO RELATED APPLICATIONS

This application claims the benefit of U.S. provisional application Ser. No. 63/436,301 filed Dec. 30, 2022, the disclosure of which is hereby incorporated in its entirety by reference herein.

### TECHNICAL FIELD

In at least one aspect, the present invention relates to thin film endovascular electrode arrays.

### BACKGROUND

There are a variety of methods used to record electrical signals or stimulate tissue in the body. To achieve high resolution recording and stimulation, small electrodes must be placed within millimeters of the target tissue in order to obtain access to nearby neurons within approximately 100 microns. In this scenario, recordings with single cell resolution are possible. In cases where the target tissue is not easily accessible (i.e. not on the surface of an organ), implantation of the electrodes to the desired location requires a highly invasive and damaging surgical procedure. Implantation entails advancing electrodes attached to wires into the tissue (e.g., neural) until the desired target is reached. Healthy tissues, including vasculature in the path of the surgical trajectory, can be injured and displaced, disturbing their structure and integrity and typically leading to an undesirable immune response to the injury and the presence of a foreign object in the tissue. If the electrodes are not accurately located adjacent to the target tissue during implantation, the procedure must be repeated, causing more damage to the body.

One example of this issue is in surgical intervention for refractory epilepsy. In order to locate the exact source of epileptiform activity in the brain tissue, stereoelectroencephalography (SEEG) probes are inserted into the brain tissue in regions where the seizures are thought to be originating. Typical SEEG probes are 0.86-1.27 mm in diameter by 10-20 mm in length with 4-10 electrodes located at the tip of the probe [1]. This process requires multiple SEEG probes to be implanted concurrently, traveling along straight trajectories to reach the target and causing significant damage to the brain. This results in permanent neurological damage in 12-18% of cases and death in 1-2% of cases [2]. In addition, the electrode sites on the probes are often not in close enough proximity to the diseased tissue, leading to inadequate seizure foci localization in 60-70% of patients [3].

In order to access tissue throughout the body in a much less invasive and damaging way, the body's network of vasculature, which exists throughout its tissues, can be utilized. By routing a miniature electrode through the veins or arteries, most parts of the body can be accessed, and the electrode can be placed within a few millimeters of tissues of interest by using a standard catheterization technique, which only requires a single small incision. In addition, if the device is implanted too far from the target tissue, it can be moved to a new location via the vasculature without causing any additional tissue damage. Since neural tissues are highly vascularized, this method of access only requires electrodes that are small enough and sufficiently precise for catheter navigation. This method of transvascular recording and stimulation has been developed and utilized in the heart and brain (the most common targets for electrical stimulation and recording) with demonstrated clinical benefit despite the greater distance from targeted tissues in the case of the brain. However, to date, the majority of devices are sized to fit into large blood vessels (>1 mm diameter), limiting their reach in the body [4, 5, 14, 6-13]. Those devices that are small enough to travel through smaller vessels (<1 mm diameter) contain very few electrodes (<5), limiting the amount of data that can be collected from or transmitted to the tissue [11,14-20]. This demonstrates the feasibility of the overall endovascular electrode array concept but current methods are unable to determine the achievable resolution of electrical recording and stimulation across the vessel wall to neighboring neural tissues. By utilizing microfabrication techniques, devices capable of higher resolution recording and stimulation (multiple electrodes packed into a small area) in small blood vessels (<1 mm diameter) can be built.

Accordingly, there is a need for improved thin film endovascular electrode arrays that can be inserted in a subject's blood vessels.

### SUMMARY

In at least one aspect, an endovascular device is provided. The endovascular device includes a thin film polymer strip having an electrode array. The thin film polymer strip includes a thin film polymer shaped into a strip having a distal end and a proximal end and a plurality of exposed electrodes positioned at the distal end of the thin film polymer. Each metal electrode is composed of a metal film. The thin film polymer strip also includes a plurality of bond pads positioned at the proximal end of the thin film polymer. A plurality of insulated traces connect each electrode to a single bond pad. The endovascular device also includes insulated micro-wires having a distal wire end electrically connected to bond pads of the thin film polymer strip and a proximal wire end stripped of insulation configured to connect to recording or stimulation equipment. Characteristically, the thin film polymer strip has a helical or cylindrical shape configured to fit around a commercial vascular guide wire.

In another aspect, the thin film polymer can be deposited by a chemical vapor deposition process.

In another aspect, the thin film polymer is Parylene C.

In another aspect, the exposed electrodes are composed of an electrically conductive metal, an electrically conductive metal alloy, or an electrically conductive metal oxide.

In another aspect, the thin film polymer electrode array is a straight strip wrapped into a helix.

In another aspect, the thin film polymer electrode array is an angled strip wrapped into two helices with different wrapping angles.

In another aspect, the thin film polymer electrode array is a sawtooth strip wrapped around a cylindrical mandrel.

In another aspect, the electrodes are functionalized for biosensing.

In another aspect, the polymer electrode array includes other embedded electrodes or sensor types.

In another aspect, the other embedded electrodes or sensor types include a component selected from the group consisting of resistive temperature or strain sensors with no exposed metal areas and interdigitated electrodes with or without exposed metal areas.

In another aspect, the electrode array is permanently attached to a commercial guide wire.

In another aspect, the device is temporarily attached to a commercial guide wire using biodissolvable adhesive; and once implanted, the adhesive is dissolved, detaching the device from the guide wire and allowing removal of the guide wire.

In another aspect, an adapter connects the device to external recording or stimulation equipment (transcutaneous use) such that one side of the adapter contains discrete connections to the proximal end of each insulated micro-wire and the other side of the adapter connects to the external recording equipment via a standard connector.

In another aspect, the proximal end of each micro-wire is connected to implantable electronics such that the endovascular device and electronics are fully implanted.

In another aspect, an endovascular recording and stimulation device that has both a small diameter (sized to navigate blood vessels <1 mm) and multiple electrode sites (>5) is provided.

In another aspect, the use of a flexible, polymer backbone that enables the device to navigate small, tight bends in vasculature is provided. Advantageously, the backbone supports the electrode sites and wiring needed to address each site.

In another aspect, the use of thin film fabrication technology, which allows for a high electrode count on a small device footprint, is provided.

In another aspect, a minimally invasive alternative to SEEG (depth) probes is provided.

In another aspect, the devices described herein can be removed and re-implanted without damaging surrounding tissue, resulting in better targeting accuracy and minimal tissue damage

In another aspect, the devices described herein can be configured in a variety of ways to be used acutely or chronically.

In another aspect, the devices described herein can be scaled up or down (within limits of fabrication techniques) to accommodate different blood vessel sizes.

In another aspect, an endovascular electrode array for high resolution, minimally invasive electrical recording, and stimulation of the nervous system and, in general, electrogenic cells and tissue is provided. High-resolution electrical recording and stimulation are accomplished by implanting small electrodes very close to the target tissue (within a few millimeters), typically traveling through healthy neural tissues, vessels, and other structures. As such, most devices that are used for such a purpose require a highly invasive surgical procedure to insert the device through healthy tissue to reach the targeted tissue, causing injury to any tissue along the device's trajectory. Since the body is highly vascularized, an endovascular approach (inserting electrodes through the network of blood vessels in the body) is a minimally invasive alternative that can be used to access most areas of the body without causing damage to the targeted electrogenic cells and tissue as well as adjacent cells and tissue. When navigating to parts of the body that are not adjacent to large blood vessels (such as regions deep within the brain), the endovascular approach requires sub-millimeter sized, flexible devices to navigate into the smaller veins and arteries. Such devices, thus far, only contain a few electrodes (less than 5) because of the size, material, and machining constraints of the traditional fabrication methods employed. To overcome these limitations, micromachining can be used to produce thin film, flexible, multi-electrode arrays suitable for endovascular deployment into sub-millimeter sized blood vessels. Miniaturization of the electrode array enables an increase in the number of electrodes as well as a final device size that permits access to most locations in the body via the vascular system and allows for medical management via electrical recording and stimulation of diseases and conditions that affect the nervous system.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be made to the following detailed description, read in conjunction with the following drawings, wherein like reference numerals denote like elements and wherein:

FIGS. 1A and 1B. The implanted portion of the full device assembly in two configurations: (A) permanently mounted on a guide wire and (B) self-expanding. Each assembly includes the flexible electrode array with outward-facing exposed electrodes for recording or stimulation and insulated wires attached to the bond pads on the array. The mounted device (A) includes a commercial guide wire.

FIGS. 2A, 2B, 2C, and 2D. The assembly process for the device (beginning with the fabricated thin film electrode array). A) A flat, thin film electrode array is fabricated (see FIG. 10 for details) with electrode sites (circles) on one end and bond pads (rectangles) on the other end. Bond pads are placed at an angle for easier wire attachment after thermoforming. B) The electrode array is wrapped around a mandrel and thermoformed to produce a helical shape. Note that the bond pads are parallel to the axis of the helical array. C) Wires are attached to the bond pads on the array. This step can be performed prior to thermoforming if the bonding method requires a flat surface. D) The device is mounted on a guide wire.

FIG. 3. Straight (left) and angled (right) electrode arrays wound around a guide wire. By using an angled array, the array can be wound around the guide wire at a shallow angle (30 degrees shown here) on the electrode end of the device, producing close proximity of all electrodes, and a steep angle (60 degrees shown here) on the bond pad end, producing wider bond pad spacing.

FIG. 4. Straight (left) and sawtooth (right) electrode arrays wound around a guide wire. By using a sawtooth array, the array can be wound around the guide wire along its long axis, resulting in a simpler fabrication process.

FIG. 5. Sawtooth electrode arrays without (left) and with (right) overlap. Overlapping areas of the polymer can be attached together, producing a more robust device.

FIG. 6. Example of straight, sawtooth, and angled electrode arrays of the same length in flat configuration (left) and wound into shape around a guide wire (right). The flat electrode arrays are shown at the same length when flat (representing the maximum dimension as allowed by the carrier wafer) but produce devices of varying length when wound into their final shape.

FIG. 7. Photos of an electrode array in the (top) straight, (middle) sawtooth, and (bottom) angled configurations while flat (prior to wrapping and thermoforming).

FIGS. 8A, 8B, and 8C. Renderings of an electrode array in the (A) straight, (B) sawtooth, and (C) angled configurations after the arrays have been wrapped and thermoformed into their final shape.

FIGS. 9A and 9B. A) Fully implantable and B) transcutaneous configurations of the device. The implantable configuration includes fully implanted electronics to record from or stimulate via the electrodes. The transcutaneous configuration includes a port at the vascular access point through which the device can directly connect to external electronic recording or stimulation equipment.

FIG. 10. Thin film fabrication process. A-E show cross sectional views of the thin film array during fabrication at the location indicated on the left image. The electrode sites can be further modified to improve electrochemical impedance or for biosensing during step D or after E. A) A substrate (silicon wafer) is coated in a thin layer of polymer (Parylene C). An optional release layer may be used in between to facilitate the release of the polymer film. B) A thin film of metal (platinum with titanium adhesion layer) is deposited and patterned using photolithography and lift-off or etching processes. C) An insulating polymer layer (Parylene C) is deposited on top of the metal. D) The polymer layers are patterned and etched using photolithography and reactive ion etching (RIE) to remove the insulation on top of the electrodes and bond pads and to cut out the outer edge of the device. E) The device is lifted off the substrate.

FIG. 11. Implantation process for a device anchored to a guide wire. The blood vessel is shown in left angling cross-hatching (with a cutout to visualize the process), the catheter is shown in intersecting cross-hatching lines (with a cutout to visualize the process), the guide wire is shown in right angling cross-hatching, and the flexible electrode array is shown in white with black electrodes. A) Catheter and guide wire are routed to the target location, and the device (permanently mounted on the guide wire) is advanced to the distal tip of the catheter. B) The catheter is slowly withdrawn (in the direction of the arrow). C) The catheter is fully withdrawn from the body, and device implantation is complete.

FIG. 12. Implantation process for a releasable device. The blood vessel is shown in left angling cross-hatching (with a cutout to visualize the process), the catheter is shown in intersecting cross-hatching lines (with a cutout to visualize the process), the guide wire is shown in right angling cross-hatching, and the flexible electrode array is shown in white with black electrodes. A) Catheter and guide wire are routed to the target location, and the device (temporarily mounted on the guide wire) is advanced to the distal tip of the catheter. B) The catheter is slowly withdrawn (in the direction of the arrow), allowing the device to expand to the inner diameter of the blood vessel. C) The catheter is fully withdrawn from the body, leaving the device in place with the guide wire at the center of the vessel. D) The guide wire is slowly withdrawn (in the direction of the arrow), leaving the device in place. E) The guide wire is fully withdrawn from the body, and device implantation is complete.

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

When referring to a numerical quantity, in a refinement, the term “less than” includes a lower non-included limit that is 5 percent of the number indicated after “less than.” A lower non-includes limit means that the numerical quantity being described is greater than the value indicated as a lower non-included limited. For example, “less than 20” includes a lower non-included limit of 1 in a refinement. Therefore, this refinement of “less than 20” includes a range between 1 and 20. In another refinement, the term “less than” includes a lower non-included limit that is, in increasing order of preference, 20 percent, 10 percent, 5 percent, 1 percent, or 0 percent of the number indicated after “less than.”

In the examples set forth herein, concentrations, temperature, and reaction conditions (e.g., thin film deposition conditions, flow rates, etc.) can be practiced with plus or minus 50 percent of the values indicated rounded to or truncated to two significant figures of the value provided in the examples. In a refinement, concentrations, temperature, and reaction conditions (e.g., thin film deposition conditions, flow rates, etc.) can be practiced with plus or minus 30 percent of the values indicated rounded to or truncated to two significant figures of the value provided in the examples. In another refinement, concentrations, temperature, and reaction conditions (e.g., thin film deposition conditions, flow rates, etc.) can be practiced with plus or minus 10 percent of the values indicated rounded to or truncated to two significant figures of the value provided in the examples.

For any device described herein, linear dimensions and angles can be constructed with plus or minus 50 percent of the values indicated rounded to or truncated to two significant figures of the value provided in the examples. In a refinement, linear dimensions and angles can be constructed with plus or minus 30 percent of the values indicated rounded to or truncated to two significant figures of the value provided in the examples. In another refinement, linear dimensions and angles can be constructed with plus or minus 10 percent of the values indicated rounded to or truncated to two significant figures of the value provided in the examples.

With respect to electrical devices, the term “connected to” means that the electrical components referred to as connected to are in electrical communication. In a refinement, “connected to” means that the electrical components referred to as connected to are directly wired to each other. In another refinement, “connected to” means that the electrical components communicate wirelessly or by a combination of wired and wirelessly connected components. In another refinement, “connected to” means that one or more additional electrical components are interposed between the electrical components referred to as connected to with an electrical signal from an originating component being processed (e.g., filtered, amplified, modulated, rectified, attenuated, summed, subtracted, etc.) before being received to the component connected thereto.

The term “electrical communication” means that an electrical signal is either directly or indirectly sent from an originating electronic device to a receiving electrical device. Indirect electrical communication can involve processing of the electrical signal, including but not limited to, filtering of the signal, amplification of the signal, rectification of the signal, modulation of the signal, attenuation of the signal, adding of the signal with another signal, subtracting the signal from another signal, subtracting another signal from the signal, and the like. Electrical communication can be accomplished with wired components, wirelessly connected components, or a combination thereof.

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within ±0%, 0.1%, 0.5%, 1%, 2%, 3%, 4%, 5% or 10% of the value or relative characteristic.

The term “electrical signal” refers to the electrical output from an electronic device or the electrical input to an electronic device. The electrical signal is characterized by voltage and/or current. The electrical signal can be stationary with respect to time (e.g., a DC signal) or it can vary with respect to time.

It should be appreciated that in any figures for electronic devices, a series of electronic components connected by lines (e.g., wires) indicates that such electronic components are in electrical communication with each other. Moreover, when lines directed connect one electronic component to another, these electronic components can be connected to each other as defined above.

Throughout this application, where publications are referenced, the disclosures of these publications in their entirety are hereby incorporated by reference into this application to more fully describe the state of the art to which this invention pertains.

“SEEG” means stereoelectroencephalography.

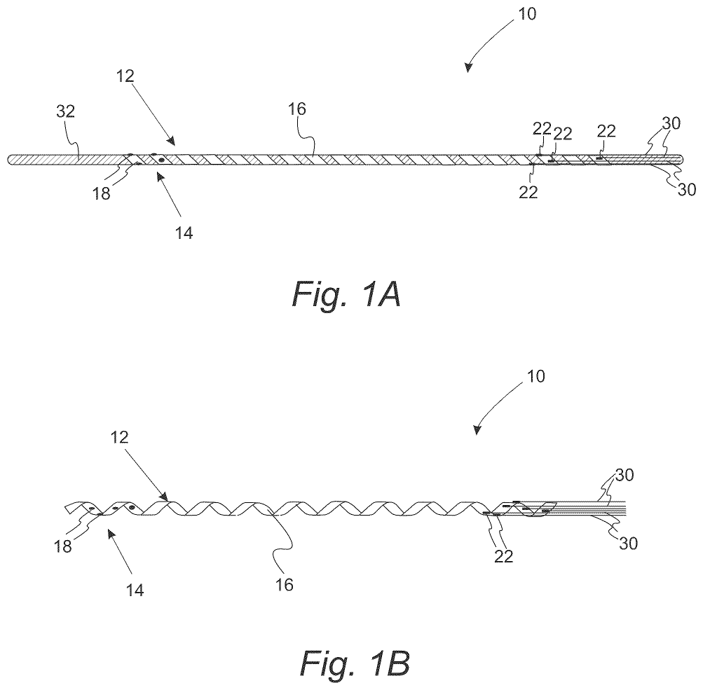

In at least one aspect, an endovascular electrode array capable of recording and stimulation in small blood vessels (<1 mm in diameter) with multiple electrodes is constructed using thin film microfabrication techniques with a flexible thin film polymer backbone. Referring to FIGS. 1A and 1B, schematics of an endovascular device are provided. Endovascular device 10 includes a thin film polymer strip 12 having an electrode array 14 disposed thereon. Thin film polymer strip 12 includes thin film polymer 16 and a plurality of exposed electrodes 18 (i.e., electrode array 14). Typically, electrode array 14 includes at least 5 exposed electrodes. In a refinement, electrode array 14 includes at least 1, 5, 6, 7, 8, 9, or 10 exposed electrodes. Although endovascular device 10 is not particularly limited by the number of exposed electrodes, typically, the number of exposed electrodes is at most 35, 32, 25, 20, 18, 16, or 15. In a refinement, a thin film polymer strip 12 and/or a thin film polymer 16 has a thickness less than or equal to 30 μm, 20 μm, 15 μm, or 10 μm and greater than or equal to 2 μm, 3 μm, 4 μm, 5 μm, or 6 μm. Typically, thin film polymer 16 is composed of polymer, and in particular, a thermoplastic polymer. Particularly useful polymers can be deposited by a chemical vapor deposition process. Examples of useful polymers for thin film polymer 16 include Parylene C, Parylene N, Parylene D, Parylene HT, polytetrafluoroethylene, polyimides, and the like. In particular, useful polymers are biocompatible polymers suitable for implantation in the body.

In another aspect, exposed electrodes 18 are composed of an electrically conductive metal, an electrically conductive metal alloy, or an electrically conductive metal oxide. For example, thin film metal electrodes 18 can be composed of platinum, palladium, iridium, rhodium, ruthenium, gold, or alloys thereof. Examples of metal oxides for exposed electrodes 18 include iridium oxides, doped tin oxides, indium tin oxide, and the like. In a refinement, exposed electrodes 18 have a diameter from about 30 to 300 μm. In a further refinement, exposed electrodes 18 have a thickness from about 50 to 1000 nm.

In another aspect, thin film polymer strip 12 and, therefore, thin film polymer 16 have a helical or cylindrical shape to fit around a commercial vascular guide wire. In this context, a cylindrical shape includes any shape that can wrap around a rod-shaped object.

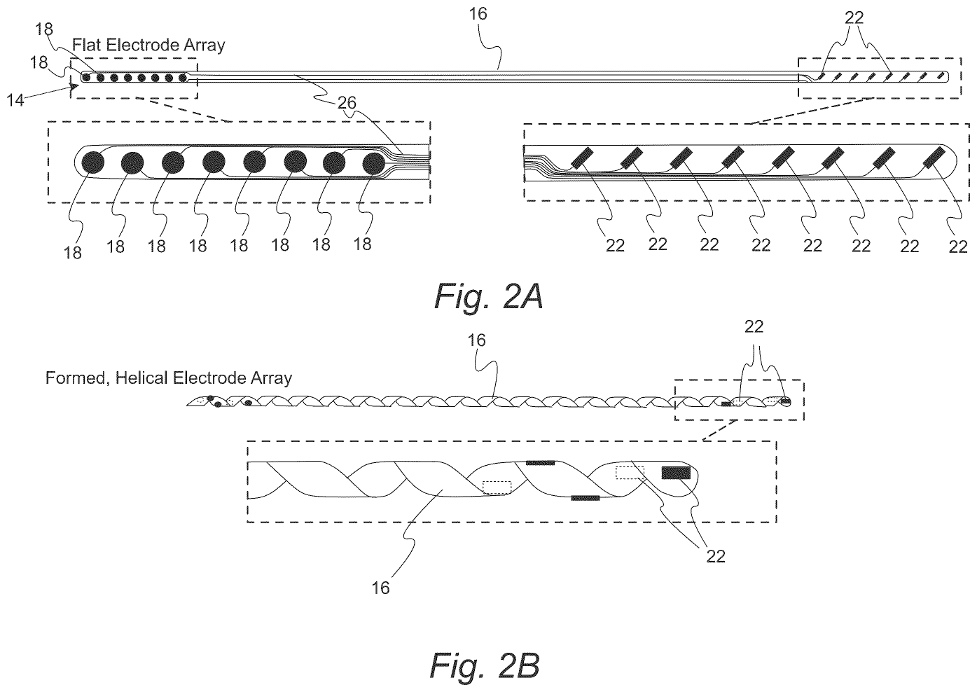

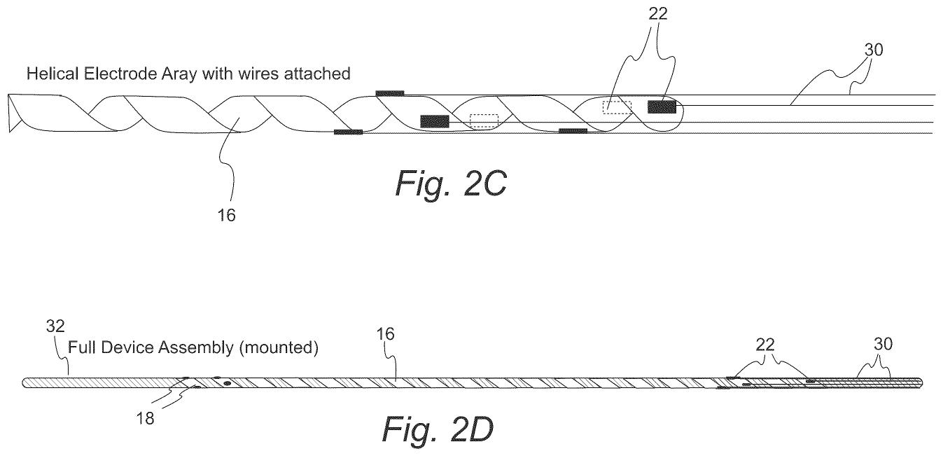

In another aspect, exposed electrodes 18 are exposed electrodes on the distal end of the endovascular device 10. In the context of the endovascular device distal and proximal refer to positions with respect to the insertion of the endovascular device. In this regard, distal means further from the recording electronics than proximal. Exposed bond pads 22 are positioned on the proximal end of endovascular device 10. Insulated traces 26 (see FIG. 2) are configured to connect each electrode 18 to a single bond pad 22. Advantageously, thin film polymer 16 and, therefore, thin film polymer strip 12 are configured with a helical or cylindrical shape to fit around a commercial vascular guide wire. Although not limited by the size of the guide wire, useful guide wires can have a diameter from 0.1 to 1.5 mm. Therefore, the helical or cylindrical shape can have a diameter in this range.

Still referring to FIGS. 1A and 1B, the distal end of the plurality of insulated wires 30 are electrically connected to bond pads 22 of the thin film polymer strip. In particular, insulated wires 30 are insulated micro-wires 30. In a refinement, a micro-wire has a diameter from about 0.5 to 100 microns and typically, a diameter from about 1 to 50 microns. The proximal end of insulated wires 30 is stripped of insulation and connected to recording or stimulation equipment.

In the variation depicted in FIG. 1A, thin film polymer strip 12 is permanently mounted on a guide wire 32. In contrast, FIG. 1B depicts a variation in which thin film polymer strip 12 is not mounted on a guide wire. In a refinement, the electrodes are typically exposed on the exterior of the helix/cylinder so as to be closer to the vessel wall. In another refinement, the electrodes can be on the “inside” the helix/cylinder in order to be wrapped around peripheral nerves.

Although endovascular device 10 can be of any length, useful lengths for endovascular device 10 and/or thin film polymer strip 12 are from 5 mm to 30 mm. In some refinements, thin film polymer strip 12 has a width from about 0.05 mm to 0.5 mm or more.

Referring to FIGS. 2A, 2B, 2C, and 2D, a method for making endovascular device 10 is schematically illustrated. Endovascular device 10 is built by first constructing a flat, long, narrow strip of flexible polymer 16 with electrodes 18 along its length at one end. Bond pads 22 for external electrical connections are positioned at the opposite end (FIG. 2A). Each bond pad is connected to a single electrode via an insulated trace 26 that runs the length of the device. The flat electrode array is formed into a helical shape with the electrodes 18 facing outward (FIG. 2B). Alternatively, electrodes 18 can face inward. Insulated wires 30 are attached to the bond pads to connect the electrode array to the recording or stimulating electronics (FIG. 2C). The entire device is wrapped around a commercial guide wire 32 with permanent or biodissolvable adhesive (FIG. 2D). Examples of biodissolvable adhesives include but are not limited to, polyethylene glycol, silk, carboxymethylcellulose, various sugars, and the like.

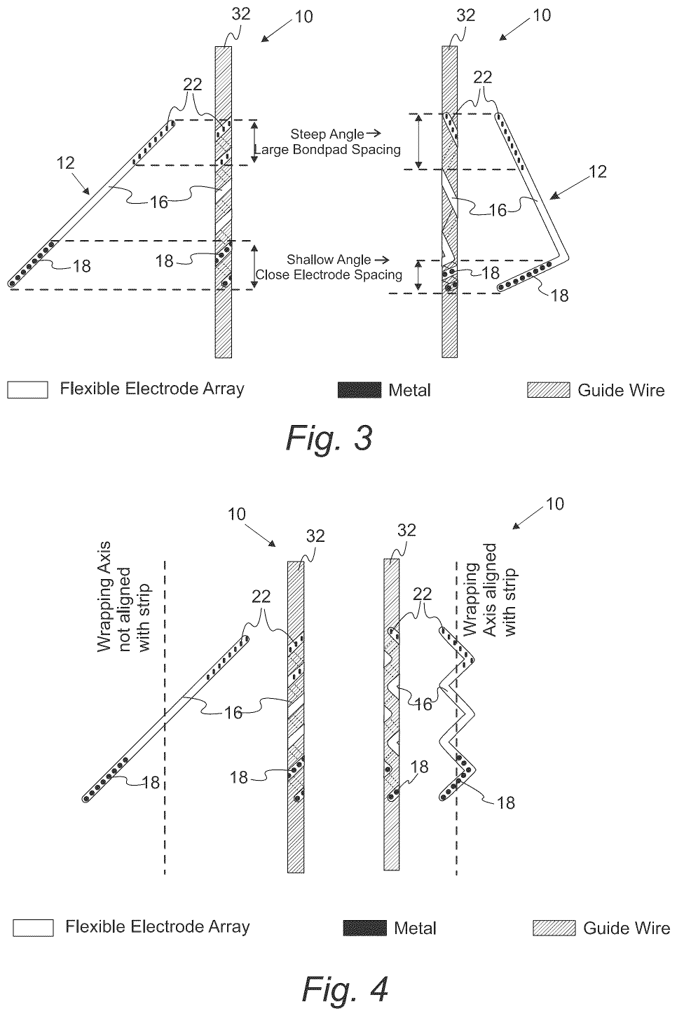

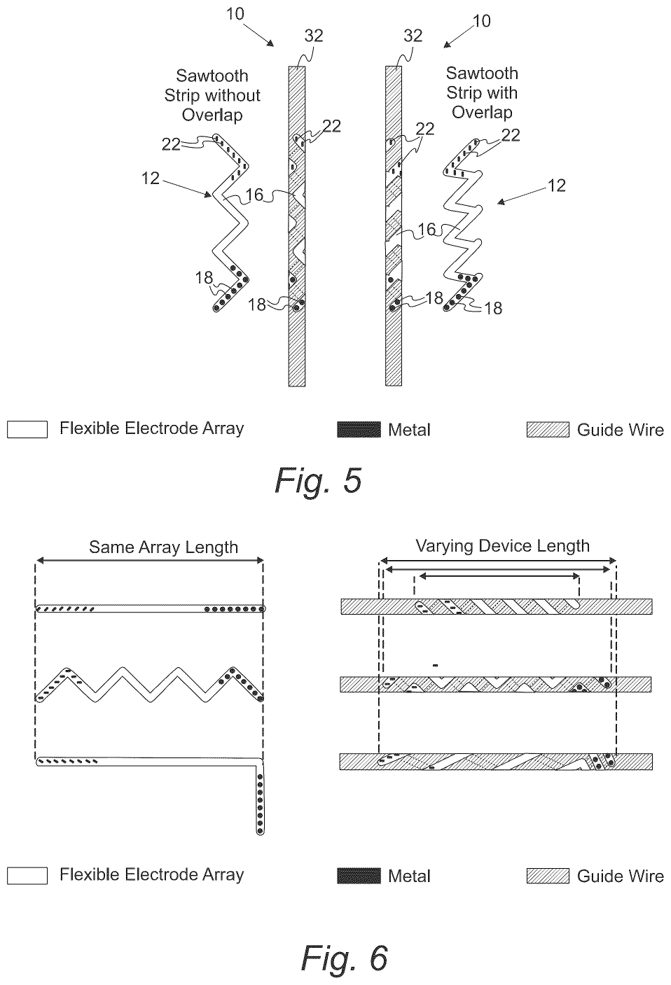

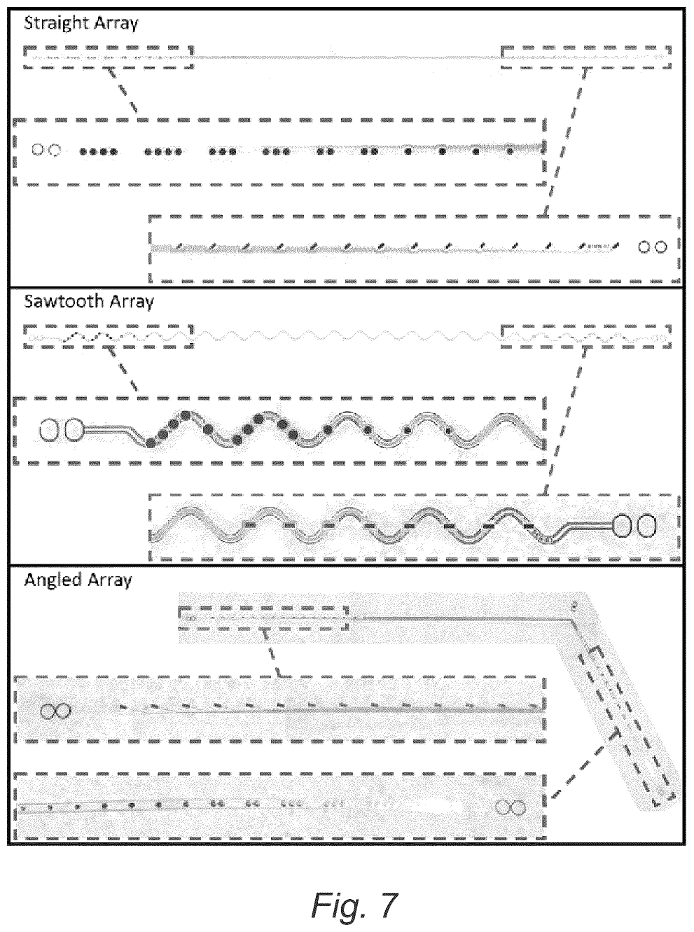

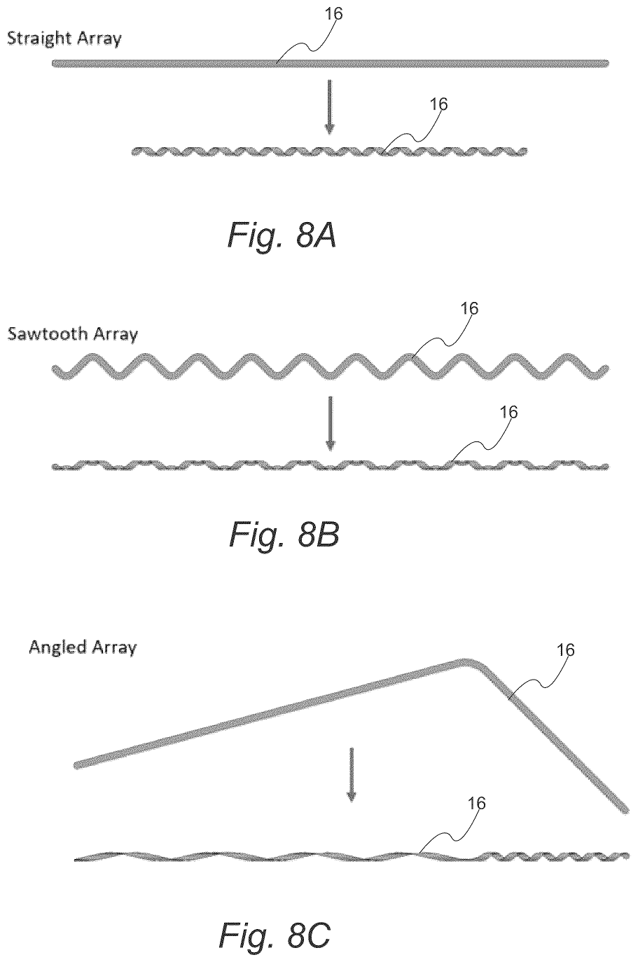

Referring to FIGS. 4, 5, and 6, alternate configurations of endovascular device 10 are provided. In these configurations, the flexible polymer strip 12 is built with bends along its length or is otherwise structured to facilitate its application or further processing. One example configuration includes a single bend in the polymer strip to produce an angled electrode array. In a refinement, this single bend can produce a winding angle from 15 to 90 degrees. In a further refinement, this single bend can produce a winding angle from 30 to 80 degrees. In this configuration, each straight portion of the polymer strip is formed into a helix. However, the two helices can be wound at different angles. This allows for each end of the device to have different functional properties. For example, as depicted in FIG. 3, a shallow-angled helix on the electrode end produces close electrode spacing and higher density recording/stimulation, and a steep-angled helix on the bond pad end increases the space between the bond pads 22. Another configuration is to build the polymer strip 16 in a sawtooth pattern. This configuration facilitates simpler processing as the polymer strip can be bent into shape along its long axis rather than being wrapped into a helix (illustrated in FIG. 4). Additionally, the sawtooth pattern can have polymer regions that overlap and bond together, making the assembled device more robust (illustrated in FIG. 5). Both of these alternate configurations can also produce a longer assembled device while using the same size carrier wafer for the thin film processing steps (see FIG. 6 for an illustration of this effect). In general, because of the nature of planar microfabrication, the electrode array is initially flat and can be modified afterward using forming techniques to impart a final shape that is compatible with deployment via standard catheterization methods. One material that can achieve this final shape is Parylene C, a thermoplastic polymer that can be molded under a thermal treatment to take on a different shape [21,22]. Examples of devices in straight, angled, and sawtooth configurations built with Parylene C are shown in the planar and formed configurations in FIG. 7 and FIG. 8 respectively.

For example, the device can be designed to wrap around a 0.25 mm guide wire with electrodes up to 200 μm in diameter. The device features can be scaled to fit a smaller or larger guide wire as long as the device dimensions stay within the limits of microfabrication techniques and material properties. The device can either be permanently anchored to the guide wire (requiring the guide wire to remain in place for the duration of recording or stimulation) or it can be temporarily anchored and released once it is in place (allowing the guide wire to be removed), as shown in the top and bottom of FIG. 1 respectively. This allows for acute or chronic implantation of the device depending on the configuration and the choice of interfacing electronics.

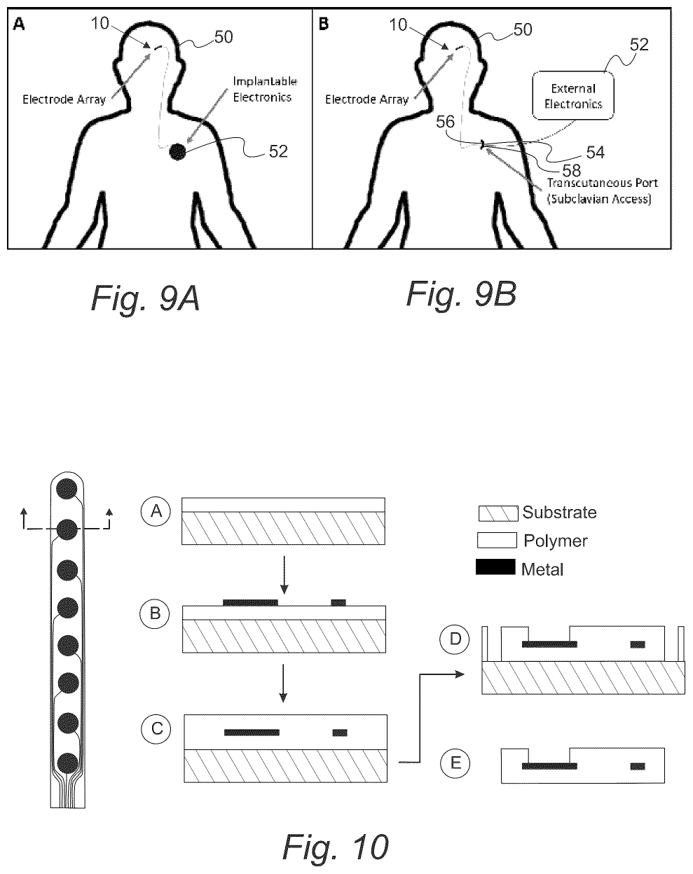

FIGS. 9A and 9B provide schematics of a system using the endovascular device described above. Endovascular device 10 is implanted in a subject 50. Endovascular device 10 is in electrical communication with an implantable pulse generator or recording system 52. If an implantable pulse generator or recording system is used, the entire system can be fully implantable (FIG. 9A). In a refinement, the proximal end of each micro-wire is connected to implantable electronics; and the device and electronics are fully implanted. Alternatively, if external equipment is used, the system is transcutaneous (FIG. 9B) or percutaneous. In a refinement, adapter 54 connects the endovascular device 10 to external recording or stimulation equipment (e.g., transcutaneous use). Adapter 54 has a first side 56 that includes discrete connections to the proximal end of each insulated micro-wire and a second side 58 that connects to the external recording equipment 52 via a standard connector.

Endovascular device 10 should be built with materials that have a long history of use in the body. The electrode array is built out of a flexible, thin film polymer backbone that is biocompatible and can be fabricated using micromachining and thermoforming techniques, such as Parylene C. Any biocompatible metal which can be selectively patterned using photolithographic methods or otherwise selectively deposited, such as gold or platinum, can be used for the electrodes, traces, and bond pads on the thin film electrode array. For stimulating devices, a coating can be added to the electrodes (such as platinum/iridium) to allow greater charge storage capacity and prevent damage to the device at normal stimulation levels. Additionally, one or more or all of the electrodes can be functionalized for selective biosensing of neurotransmitters, drugs, or other molecules of interest. The insulated wires are made of any biocompatible metal and coated with a robust biocompatible polymer such as polyimide or polytetrafluoroethylene (PTFE) to prevent electrical leakage. The insulated wires can be attached to the bond pads using a variety of techniques and materials, such as attachment with conductive epoxy or anisotropic conductive film, or ultrasonic wire bonding.

In another embodiment, a method for using the endovascular device set forth herein is provided. The method includes a step of inserting an endovascular device into a subject, the endovascular device comprising a thin film polymer strip having an electrode array. The design of the thin film polymer strip is described above. Readings from the endovascular device are recorded or the subject is stimulated with the endovascular device.

The following examples illustrate the various embodiments of the present invention. Those skilled in the art will recognize many variations that are within the spirit of the present invention and the scope of the claims.

Referring to FIG. 10, the endovascular electrode array described above was fabricated using a thin film Parylene C backbone, platinum conductors, and polyimide wire insulation. First, 4 μm of Parylene C was deposited onto a silicon carrier wafer. 200 nm of platinum (with 15 nm titanium adhesion layer on both sides) was deposited and patterned on top of the base Parylene C layer to define 8 to 16 electrodes ranging in size from 50 to 200 μm, then insulated with 4 μm of Parylene C. Electrodes and bond pads were exposed and the outline of the array cut out using a switched chemistry reactive ion etch [23]. Other selective etching or cut-out processes could also be used. The overall process is summarized in FIG. 10.

Each array was removed from the carrier wafer and mounted on a Teflon block using Kapton tape. Insulated wires were cut to the desired length, stripped of insulation for approximately 1 mm on the tip, and attached to the bond pads on the array using conductive silver epoxy. Exposed portions of the wire and epoxy were insulated using a thin, insulating epoxy. After wires were attached, the device was removed from the Teflon block, wrapped around a 0.25 mm diameter mandrel to produce the desired shape (FIG. 6 shows the resulting shapes for straight, sawtooth, and angled arrays), and thermoformed at 100° C. for 12 hours. After cooling, the device was removed from the mandrel and coated with 2 μm of Parylene C to ensure full insulation of the bond pads and wire connections. Electrodes were covered during the Parylene insulation process to prevent any blockage of the electrical contacts. Once insulated, the device was wrapped around a 0.25 mm diameter guide wire and held in place using small amounts of insulating epoxy at the two ends and every few millimeters along the length of the thin film array. The insulated wires were loosely wrapped around the guide wire for easier handling. Devices were tested throughout the fabrication process and after fabrication was complete to verify the functionality of the electrodes. In a variation, wires are attached after thermoforming without 2 um Parylene C insulation.

As discussed previously, the dimensions of the device can be altered to fit onto a different sized guide wire and the electrode sizes can be altered to meet the recording or stimulation specifications. In addition, the insulated wires can be attached using methods such as anisotropic conductive film or ultrasonic wire bonding. Finally, the electrode array can be thermoformed to a larger dimension (to match the diameter of the target vein) and be temporarily attached to the guide wire with biodissolvable adhesive if the application requires the removal of the wire. Several biodissolvable adhesives are commonly used in neural probe applications, such as polyethylene glycol, silk, carboxymethylcellulose, or various sugars [1].

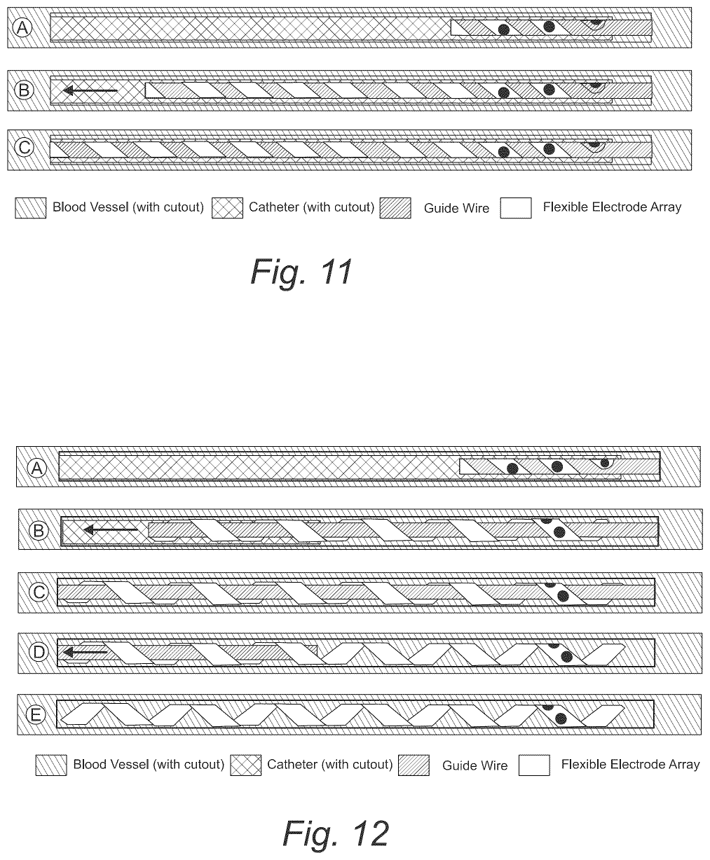

Once fully constructed, the device is implanted using a standard catheterization technique. Briefly, a sheath is placed through the skin and into the blood vessel at the access point and a microcatheter is routed through the vasculature to the target location. The specific device described in this section is designed to target the hippocampal vein using the subclavian or internal jugular vein as the access point. After the catheter has reached the target, the device/guide wire assembly is inserted into the catheter until the electrode array reaches the catheter tip. The catheter is then withdrawn to expose the electrodes to the surrounding tissue. For applications which require the removal of the guide wire, the biodissolvable adhesive is dissolved as the catheter is withdrawn, allowing the device to self-expand into place. Once expanded, the guide wire is withdrawn. The implantation process for devices which are mounted on the guide wire and self-expanding devices are illustrated in FIG. 11 and FIG. 12 respectively.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

[1] Thielen B and Meng E 2021 A comparison of insertion methods for surgical placement of penetrating neural interfaces J. Neural Eng. 18 041003
[2] Jobst B C and Cascino G D 2015 Resective epilepsy surgery for drug-resistant focal epilepsy: A review JAMA—J. Am. Med. Assoc. 313 285-93
[3] McGonigal A, Bartolomei F, Régis J, Guye M, Gavaret M, Fonseca A T Da, Dufour H, Figarella-Branger D, Girard N, Péragut J C and Chauvel P 2007 Stereoelectroencephalography in presurgical assessment of MRI-negative epilepsy Brain 130 3169-83
[4] Oxley T J, Opie N L, John S E, Rind G S, Ronayne S M, Wheeler T L, Judy J W, McDonald A J, Dornom A, Lovell T J H, Steward C, Garrett D J, Moffat B A, Lui E H, Yassi N, Campbell B C V, Wong Y T, Fox K E, Nurse E S, Bennett I E, Bauquier S H, Liyanage K A, Van Der Nagel N R, Perucca P, Ahnood A, Gill K P, Yan B, Churilov L, French C R, Desmond P M, Horne M K, Kiers L, Prawer S, Davis S M, Burkitt A N, Mitchell P J, Grayden D B, May C N and O'Brien T J 2016 Minimally invasive endovascular stent-electrode array for high-fidelity, chronic recordings of cortical neural activity Nat. Biotechnol. 34 320-7
[5] Takahata K, Gianchandani Y B and Wise K D 2006 Micromachined antenna stents and cuffs for monitoring intraluminal pressure and flow J. Microelectromechanical Syst. 15 1289-98
[6] Oxley T J, Opie N L, John S E, Rind G S, Ronayne S M, Wheeler T L, Judy J W, McDonald A J, Dornom A, Lovell T J H, Steward C, Garrett D J, Moffat B A, Lui E H, Yassi N, Campbell B C V, Wong Y T, Fox K E, Nurse E S, Bennett I E, Bauquier S H, Liyanage K A, Van Der Nagel N R, Perucca P, Ahnood A, Gill K P, Yan B, Churilov L, French C R, Desmond P M, Horne M K, Kiers L, Prawer S, Davis S M, Burkitt A N, Mitchell P J, Grayden D B, May C N and O'Brien T J 2016 Supplementary Figures: Minimally invasive endovascular stent-electrode array for high-fidelity, chronic recordings of cortical neural activity Nat. Biotechnol. 34
[7] Opie N L, Oxley T J, Rind G S, Ronayne S M and John S E 2020 Endovascular Device for Sensing and or Stimulating Tissue
[8] Rosero S 2015 Leadless Implantable Intravascular Electrophysiologic Device for Neurologic/Cardiovascular Sensing and Stimulation
[9] Speicher V L and Bach S M J 1986 Intravascular Multiple Electrode Unitary Catheter
[10] Mirowski M, Mower M M and Denniston R H I 1976 Cardioverting Device having Single Intravascular Catheter Electrode System and Method for its Use
[11] Lee S P, Liu C, Ghaffari R, Work J, Quivira F, Klinker L E and Hsu Y-Y 2017 Catheter Device Including Flow Sensing
[12] Dahl R W, Swanson D K and Lipson D 1993 Stent-type Defibrillation Electrode Structures
[13] Stack R S, Williams M S, Fifer D W, Glenn R A, Orth G A, Elliott L and Stack C 2015 Intravascular Electrode System and Method
[14] Hess S R and Kovacs T 1987 Percutaneous Lead having Radially Adjustable Electrode
[15] Watanabe H, Takahashi H, Nakao M, Walton K and Llinás R R 2009 Intravascular Neural Interface with Nanowire Electrode Electron. Commun. Japan 92 29-37
[16] Llinas R R, Walton K D, Nakao M, Hunter I and Anquetil P A 2005 Neuro-vascular central nervous recording/stimulating system: Using nanotechnology probes J. Nanoparticle Res. 7 111-27
[17] Nakase H, Ohnishi H, Touho H, Karasawa J, Yamamoto S and Shimizu K 1995 An Intra-Arterial Electrode for Intracranial Electro-Encephalogram Recordings Acta Neurochir. (Wien). 136 103-5
[18] Penn R D, Hilal S K, Michelsen W J, Goldensohn E S and Driller J 1973 Intravascular intracranial EEG recording Neurosurgery 38 239-43
[19] Mehra R 1991 Implantable Electrode for Location Within a Blood Vessel
[20] Tomaschko D K, Broman D J, Eidenschink T E, Hastings R and Olson R J 2009 Pacing Catheter with Stent Electrode
[21] Kim B J, Chen B, Gupta M and Meng E 2013 Three Dimensional Transformation of Parylene Thin Film Structures via Thermoforming 2013 IEEE 26th International Conference on Micro Electro Mechanical Systems (MEMS) (IEEE) pp 339-42
[22] Yoon E, Koo B, Wong J, Elyahoodayan S, Weiland J D, Lee C D, Petrossians A and Meng E 2020 An implantable microelectrode array for chronic in vivo epiretinal stimulation of the rat retina J. Micromechanics Microengineering 30 124001
[23] Meng E, Li P Y and Tai Y C 2008 Plasma removal of Parylene C J. Micromechanics Microengineering 18 045004

What is claimed is:

1. An endovascular device comprising:
a thin film polymer strip having an electrode array, the thin film polymer strip comprising:
a thin film polymer shaped into a strip having a distal end and a proximal end;
a plurality of exposed electrodes positioned at the distal end of the thin film polymer;
a plurality of bond pads positioned at the proximal end of the thin film polymer; and
a plurality of insulated traces connecting each electrode to a single bond pad; and
insulated micro-wires having a distal wire end electrically connected to bond pads of the thin film polymer strip and a proximal wire end stripped of insulation configured to connect to recording or stimulation equipment, wherein the thin film polymer strip has a helical or cylindrical shape configured to fit around a commercial vascular guide wire.

2. The endovascular device of claim 1, wherein the thin film polymer is composed of a thermoplastic polymer.

3. The endovascular device of claim 1, wherein the thin film polymer is composed of Parylene C.

4. The endovascular device of claim 1, wherein plurality of exposed electrodes are composed of a metal film.

5. The endovascular device of claim 4, wherein the metal film is composed of an electrically conductive metal, an electrically conductive metal alloy, or an electrically conductive metal oxide.

6. The endovascular device of claim 5, wherein the metal film is composed of platinum, palladium, iridium, rhodium, ruthenium, gold, or alloys thereof.

7. The endovascular device of claim 1, wherein the thin film polymer strip having an electrode array is a straight strip wrapped into a helix.

8. The endovascular device of claim 1, wherein the thin film polymer strip having an electrode array is an angled strip wrapped into two helices with different wrapping angles.

9. The endovascular device of claim 1, wherein the thin film polymer strip having an electrode array is a sawtooth strip wrapped around a cylindrical mandrel.

10. The endovascular device of claim 1, wherein one or more of the exposed electrodes are functionalized for biosensing.

11. The endovascular device of claim 1, wherein the thin film polymer strip having an electrode array includes other embedded electrodes or sensor types.

12. The endovascular device of claim 11, wherein the other embedded electrodes or sensor types include a component selected from the group consisting of resistive temperature or strain sensors with no exposed metal areas and interdigitated electrodes with or without exposed metal areas.

13. The endovascular device of claim 1, wherein the electrode array is permanently attached to a commercial guide wire.

14. The endovascular device of claim 1 wherein:
the endovascular device is temporarily attached to a commercial guide wire using a biodissolvable adhesive; and
once implanted, the biodissolvable adhesive is dissolved, detaching the endovascular device from the commercial guide wire and allowing removal of the commercial guide wire.

15. The endovascular device of claim 1, wherein an adapter connects the endovascular device to external recording or stimulation equipment (transcutaneous use), the adapter including:
a first side that includes discrete connections to the proximal end of each insulated micro-wire; and
a second side that connects to external recording equipment via a standard connector.

16. The endovascular device of claim 1, wherein:
the proximal end of each micro-wire is connected to implantable electronics; and
the endovascular device and electronics are fully implanted.

17. A method comprising:
a) inserting an endovascular device into a subject, the endovascular device comprising a thin film polymer strip having an electrode array, the thin film polymer strip comprising:
a thin film polymer shaped into a strip having a distal end and a proximal end;
a plurality of exposed electrodes positioned at the distal end of the thin film polymer;
a plurality of bond pads positioned at the proximal end of the thin film polymer; and
a plurality of insulated traces connecting each electrode to a single bond pad; and
insulated micro-wires having a distal wire end electrically connected to bond pads of the thin film polymer strip and a proximal wire end stripped of insulation configured to connect to recording or stimulation equipment, wherein the thin film polymer strip has a helical or cylindrical shape configured to fit around a commercial vascular guide wire;
d) recording readings from the endovascular device or stimulating the subject with the endovascular device.

18. The method of claim 17, wherein the thin film polymer is composed of a thermoplastic polymer and the plurality of exposed electrodes are composed of an electrically conductive metal, an electrically conductive metal alloy, or an electrically conductive metal oxide.

19. The method of claim 17, wherein the thin film polymer strip having an electrode array is a straight strip wrapped into a helix.

20. The method of claim 17, wherein the thin film polymer strip having an electrode array is an angled strip wrapped into two helices with different wrapping angles.
